(19) United States
(12) Patent Application Publication
(10) Pub. No.: US 2007/0099926 A1
(43) Pub. Date: May 3, 2007

(54) Combination therapy for treating chronic inflammatory diseases

(76) Inventors: Ian W. Rodger, Doylestown, PA (US); Sidney Mazel, Basking Ridge, NJ (US)

Correspondence Address: MERCK AND CO., INC, P O BOX 2000, RAHWAY, NJ, 07065-0907, US

(21) Appl. No.: 10/561,672

(22) PCT Filed: Jun. 28, 2004
(86) PCT No.: PCT/US04/20715

§ 371 Date: Dec. 21, 2005

Related U.S. Application Data
(60) Provisional application No. 60/484,499, filed on Jul. 2, 2003.

(51) Int. Cl. A61K 31/525; A61K 31/415; A61K 31/365

(52) U.S. Cl. 514/251; 514/406; 514/471

(57) ABSTRACT

The present inventions is directed to a novel DMARD sparing method for treating chronic inflammatory diseases or conditions, such as rheumatoid arthritis, comprising the short-term administration of a disease modifying anti-rheumatic drug (DMARD) followed by reduction of the DMARD and co-administration of a cyclooxygenase-2 selective inhibitor or cessation of the DMARD and continued maintenance therapy using a COX-2 selective inhibitor alone. The present invention provides for an effective DMARD sparing therapy in patients suffering from inflammatory diseases or conditions.

### BACKGROUND OF THE INVENTION

Disease modifying anti-rheumatic drugs (DMARDs) typified by etanercept, infliximab and methotrexate are currently used as effective therapy for chronic inflammatory diseases, such as rheumatoid arthritis. However, there are severe side effects and extremely high costs associated with these drugs. The present invention is directed to a combination therapy of a cyclooxygenase-2 selective inhibitor and a disease modifying anti-rheumatic drug for treating chronic inflammatory diseases in accordance with a specified dosing regimen. The present invention is directed to a therapeutic regimen whereby the use of a cyclooxygenase-2 selective inhibitor in combination with a disease modifying anti-rheumatic drug leads to a shorter-term use of the disease modifying anti-rheumatic drug and/or a reduction in the dose of the prescribed DMARD. Such a reduction in dose or restriction in timescale of its use will result in superior patient tolerability and reduction in associated DMARD-induced side effects and toxicity.

### SUMMARY OF THE INVENTION

The present invention is directed to a novel DMARD sparing method for treating chronic inflammatory diseases or conditions, such as rheumatoid arthritis, comprising the short-term administration of a disease modifying anti-rheumatic drug (DMARD) followed by reduction of the DMARD and co-administration of a cyclooxygenase-2 selective inhibitor or cessation of the DMARD and continued maintenance therapy using a COX-2 selective inhibitor alone. The present invention provides for an effective DMARD sparing therapy in patients suffering from inflammatory diseases or conditions.

### DETAILED DESCRIPTION OF THE INVENTION

The invention encompasses a DMARD sparing method for treating a chronic inflammatory disease or condition in a human patient in need thereof, comprising administering to the patient a therapeutically effective amount of a DMARD in accordance with a DMARD dosage regimen for a period of time, and thereafter:

(A) co-administering to the patient a therapeutically effective amount of a DMARD and a cyclooxygenase-2 selective inhibitor in accordance with a combination dosage regimen, or

(B) administering to the patient a therapeutically effective amount of a cyclooxygenase-2 selective inhibitor in accordance with a COX-2 dosage regimen, whereby the total exposure to the DMARD is reduced.

An embodiment of the invention encompasses the above method wherein the DMARD is selected from the group consisting of: methotrexate, infliximab, etanercept, leflunomide, sulfasalazine, azathioprine, cyclosporine, hydroxychloroquine and pencillamine.

Another embodiment of the invention encompasses the above method wherein the cyclooxygenase-2 selective inhibitor is selected from the group consisting of: rofecoxib, etoricoxib, celecoxib, valdecoxib, parecoxib, lumiracoxib, BMS347070, tiracoxib, ABT963, CS502 and GW406381.

Another embodiment of the invention encompasses a DMARD sparing method for treating a chronic inflammatory disease or condition in a human patient in need thereof, comprising administering to the patient a therapeutically effective amount of a DMARD in accordance with a DMARD dosage regimen for a period of time, and thereafter co-administering to the patient a therapeutically effective amount of a DMARD and a cyclooxygenase-2 selective inhibitor in accordance with a combination dosage regimen, whereby the total exposure to the DMARD is reduced. Within this embodiment of the invention, the DMARD is methotrexate. Also within this embodiment of the invention, the DMARD is methotrexate and the DMARD dosing regimen is 7.5 to 22.5 mg once weekly. Also within this embodiment of the invention, the DMARD is methotrexate, the DMARD dosing regimen is 7.5 to 22.5 mg once weekly, and the period of time in accordance with the DMARD dosage regimen is 8 weeks. Also within this embodiment of the invention, the cyclooxygenase-2 selective inhibitor is rofecoxib. Also within this embodiment of the invention, the combination dosage regimen comprises: administering rofecoxib at a dose of 12.5 or 25 mg on a once daily basis and reducing the amount of methotrexate by 2.5 mg per week relative to the DMARD dosing regimen.

In another aspect of this embodiment, the DMARD is etanercept. Also within this embodiment the DMARD is etanercept and the DMARD dosing regimen is 25 mg twice weekly. Also within this embodiment, the cyclooxygenase-2 selective inhibitor is rofecoxib. Also within this embodiment, the cyclooxygenase-2 selective inhibitor is rofecoxib and the combination dosage regimen comprises: administering rofecoxib at a dose of 12.5 or 25 mg on a once daily basis and administering etanercept at a dose of 25 mg on a once weekly basis.

In another aspect of this embodiment, the invention encompasses the above method further comprising: eliminating administering the DMARD to the patient and continuing therapy with the cyclooxygenase-2 selective inhibitor alone.

In another embodiment, the invention encompasses a DMARD sparing method for treating a chronic inflammatory disease or condition in a human patient in need thereof, comprising administering to the patient a therapeutically effective amount of a DMARD in accordance with a DMARD dosage regimen for a period of time, and thereafter administering to the patient a therapeutically effective amount of a cyclooxygenase-2 selective inhibitor in accordance with a COX-2 dosage regimen, whereby the total exposure to the DMARD is reduced. Within this embodiment the DMARD is etanercept. Also within this embodiment the DMARD is etanercept and the DMARD dosing regimen is 25 mg twice weekly. Also within this embodiment the cyclooxygenase-2 selective inhibitor is rofecoxib. Also within this embodiment, rofecoxib is administered at a dose of 12.5 or 25 mg on a once daily basis.

Another embodiment of the invention encompasses further co-administering a cyclooxygenase-2 selective inhibitor to the patient being administered the DMARD in accordance with the DMARD dosage regimen, wherein the cyclooxygenase-2 selective inhibitor is administered at a dose which, in combination with the DMARD in accordance with a DMARD dosage regimen, is effective to treat the chronic inflammatory disease or condition.

The present invention is useful for treating chronic inflammatory disease or conditions. The term “treating a chronic inflammatory disease or condition” means treating or preventing any chronic inflammatory disease or condition, such as rheumatoid arthritis, degenerative joint diseases (osteoarthritis), gout, ankylosing spondylitis and bursitis. The term “treating” encompasses not only treating a patient to relieve the patient of the signs and symptoms of the disease or condition but also prophylactically treating an asymptomatic patient to prevent the onset or progression of the disease or condition.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term includes the amount of the drug or combination of drugs that will provide patient relief from the signs and symptoms of any chronic inflammatory disease or condition. With respect to the combination dose of each agent is therapeutically effective for purposes of this invention, even though the individual dosage amounts may be sub-therapeutic amounts. Each agent at therapeutic amounts is also encompassed.

The term “patient” includes mammals, especially humans, who take a selective COX-2 inhibitor for any of the uses described herein. Administering of the drug to the patient includes both self-administration and administration to the patient by another person.

The terms “inhibitor of cyclooxygenase-2”, “cyclooxygenase-2 selective inhibitor” and “COX-2 inhibitor” as used herein embrace compounds which selectively inhibit cyclooxygenase-2 over cyclooxygenase-1, including pharmaceutically acceptable salts thereof. Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC₅₀ of less than about 2 TM in the human whole blood COX-2 assay, yet have a cyclooxygenase-1 IC₅₀ of greater than about 5 TM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects, especially erosions and ulceration of the upper gastrointestinal mucosa.

Many cyclooxygenase-2 selective inhibitors are known in the art. Examples of cyclooxygenase-2 selective inhibitors include rofecoxib (VIOXX®, see U.S. Pat. No. 5,474,995, hereby incorporated by reference in its entirety), etoricoxib (ARCOXIA™ see U.S. Pat. No. 5,861,419, hereby incorporated by reference in its entirety), celecoxib (CELEBREX®, see U.S. Pat. No. 5,466,823, hereby incorporated by reference in its entirety), valdecoxib (see U.S. Pat. No. 6,633,272, hereby incorporated by reference in its entirety), parecoxib (see U.S. Pat. No. 5,932,598, hereby incorporated by reference in its entirety), lumiracoxib (PREXIGE®, Novartis), BMS347070 (Bristol Myers Squibb), tiracoxib or JTE522 (Japan Tobacco), ABT963 (Abbott), CS502 (Sankyo) and GW406381 (GlaxoSmithKline).

The terms “disease modifying anti-rheumatic drug” or “DMARD” means compounds that relieve the symptoms of and help control chronic inflammatory diseases or conditions by modifying the actual disease process. Examples include, methotrexate, leflunomide, sulfasalazine, azathioprine, cyclosporine, hydroxychloroquine, pencillamine as well as tumor necrosis factor (TNF) antagonists, such as 2-[(4,5-dimethoxy-2-methyl-3,6-dioxo-1,4-cyclohexadien-1-yl)methylene]-undecanoic acid, lenercept, etanercept, BB-2275, PCM4, SH-636, onercept, vinigrol, TBP-1, solimastat, MIDL-201112, AGT-1; D-609, 4-[3-(cyclopentyloxy)-4-methoxyphenyl]-pyrrolidinone, CytoTAb®; and infliximab

The term “DMARD dosage regimen” means administering the DMARD to the patient in any amount that is effective to treat the chronic inflammatory disease or condition. Convention doses of DMARDs may be employed with the present invention. For example, methotrexate may be administered to a patient at a dose of 7.5 mg to 22.5 mg on a once weekly basis. Etanercept may be administered to a patient at a dose of 25 mg on a twice weekly basis for an indefinite period. Infliximab may be administered at a dose of 3 mg/kg as an intravenous infusion followed with additional similar doses at 2 and 6 weeks after the first infusion then every 8 weeks thereafter.

The term “period of time” means any period of time during which a patient is being treated in accordance with the DMARD dosage regimen. Preferably, this period of time means the period of time the patient is under treatment in accordance with the DMARD dosage regimen wherein the a patient sees maximal efficacy from the DMARD treatment. For example, the “period of time” in accordance with the DMARD dosage regimen may be 7.5 mg to 22.5 mg of methotrexate once weekly for a period of 8 weeeks.

The term “co-administering” means concomitantly or sequentially administering the agents to the patient. The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently. The term “sequentially administering” means administering the agents at separately staggered times. Thus, agents can be sequentially administered such that the beneficial pharmaceutical effect are realized by the patient at substantially the same time. Thus, for example, if a cyclooxygenase-2 selective inhibitors and other agent are both administered on a once a day basis, the interval of separation between sequential administration of the two agents can be up to twelve hours apart.

The term “combination dosage regimen” means any amount of the cyclooxygenase-2 selective inhibitor and DMARD that when co-administered, are effective to treat the chronic inflammatory disease or condition; provided, however, that total exposure to the DMARD is reduced. Conventional doses of cyclooxygenase-2 selective inhibitors and DMARDs may be used with the present invention. Such amounts are well known in the art and described, for example, in the PDR. Typically, suitable levels of a cyclooxygenase-2 selective inhibitor will be about 5 to 500 mg per day, preferably 10 to 200 mg per day, and especially 10 to 100 mg/kg per day. The cyclooxygenase-2 selective inhibitor may be administered on a regimen of up to 6 times per day, preferably 1 to 4 times per day, and especially once per day. For example, rofecoxib may be administered at a dose of 12.5 or 25 mg on a once daily basis and methotrexate may be administered at a dose that is reduced by 2.5 mg per week relative to the first dosing regimen. The term “combination dosage regimen” also includes administering the DMARD and COX-2 inhibitor once.

The term “COX-2 dosage regimen” means any amount of the cyclooxygenase-2 selective inhibitor that is effective to treat the inflammatory disease or condition. Typically, conventional dosage regimens of cyclooxygenase-2 selective inhibitors will be employed. Preferably the cycyloxygenase-2 selective inhibitor is dosed on a once a day basis. For example, rofecoxib may be employed at a dose of 12.5 mg or 25 mg on a once daily basis. The term “COX-2 dosage regimen” also includes administering a COX-2 inhibitor once.

The term “total exposure to the DMARD is reduced” means that the total amount of the DMARD administered to the patient is reduced relative to if therapy was continued with the DMARD alone in accordance with the DMARD dosage regimen. For example, the dose of methotrexate may be reduced by 2.5 mg per week if co-dosed with a cyclooxygenase-2 selective inhibitor while maintaining effective treatment for the chronic inflammatory disease or condition.

The compounds of use in this invention may have one or more chiral centers and the present compounds may occur as racemates, racemic mixtures and as individual diasteriomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

The cyclooxygenase-2 selective inhibitors and disease modifying anti-rheumatic drugs that may be used with this invention encompass all pharmaceutically acceptable salt forms of the compounds. Examples of such salt forms include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Conventional dosage forms of the DMARD and cyclooxygenase-2 selective inhibitor may be used in the present invention. However, where appropriate, other forms are also encompassed in the invention. Thus, the invention encompasses the active ingredients being administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, benzyl alcohol, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active agents for use in the present invention may also be administered in the form of a suppositories for rectal administration of the drug. These compositions can be prepared by mixing the drug with a suitable nonirritating excipient which is solid at ordinary temperatures but liquid at the rectal temperature and will therefore melt in the rectum to release the drug. Such materials are cocoa butter and polyethylene glycols.

For topical use, creams, ointments, gels, solutions or suspensions, etc., containing the compounds are employed. (For purposes of this application, topical application shall include mouth washes and gargles.) Topical formulations may generally be comprised of a pharmaceutical carrier, cosolvent, emulsifier, penetration enhancer, preservative system, and emollient.

The instant invention includes the use of both oral rapid-release and time-controlled release pharmaceutical formulations. A particular example of an oral time-controlled release pharmaceutical formulation is described in U.S. Pat. No. 5,366,738. Such pharmaceutical compositions are known to those of ordinary skill in the pharmaceutical arts; for example, see Remington's Pharmaceutical Sciences, Mack Publishing Co., Easton, PA.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The invention will now be exemplified by the following non-limiting examples:

### EXAMPLE 1

A male human patient suffering from rheumatoid arthritis is administered 22.5 mg of methotrexate sodium tablets on a once weekly basis. After 8 weeks of such therapy, the dose of methotrexate sodium tablets is reduced to 20 mg once weekly and the patient is also administered 12.5 mg of rofecoxib oral tablets on a once daily basis. The additional benefit of rofecoxib then permits the reduction in the methotrexate dose by 2.5 mg decrements to the point where DMARD activity of methotrexate is maintained without consequent, associated side-effects/intolerance. This might well result in a stable therapeutic regimen of 10 mg methotrexate in combination with 12.5 mg rofecoxib.

What is claimed is:

1. A DMARD sparing method for treating a chronic inflammatory disease or condition in a human patient in need thereof, comprising administering to the patient a therapeutically effective amount of a DMARD in accordance with a DMARD dosage regimen for a period of time, and thereafter:
(A) co-administering to the patient a therapeutically effective amount of a DMARD and a cyclooxygenase-2 selective inhibitor in accordance with a combination dosage regimen, or
(B) administering to the patient a therapeutically effective amount of a cyclooxygenase-2 selective inhibitor in accordance with a COX-2 dosage regimen,
whereby the total exposure to the DMARD is reduced.

2. The method according to claim 1 wherein the DMARD is selected from the group consisting of: methotrexate, infliximab, etanercept, leflunomide, sulfasalazine, azathioprine, cyclosporine, hydroxychloroquine and pencillamine.

3. The method according to claim 1 wherein the cyclooxygenase-2 selective inhibitor is selected from the group consisting of: rofecoxib, etoricoxib, celecoxib, valdecoxib, parecoxib, lumiracoxib, BMS347070, tiracoxib, ABT963, CS502 and GW406381.

4. A DMARD sparing method in accordance with claim 1 for treating a chronic inflammatory disease or condition in a human patient in need thereof, comprising administering to the patient a therapeutically effective amount of a DMARD in accordance with a DMARD dosage regimen for a period of time, and thereafter co-administering to the patient a therapeutically effective amount of a DMARD and a cyclooxygenase-2 selective inhibitor in accordance with a combination dosage regimen, whereby the total exposure to the DMARD is reduced.

5. The method according to claim 4 wherein the DMARD is methotrexate.

6. The method according to claim 5 wherein the DMARD dosing regimen is 7.5 to 22.5 mg once weekly.

7. The method according to claim 6 wherein the period of time in accordance with the DMARD dosage regimen is 8 weeks.

8. The method according to claim 7 wherein the cyclooxygenase-2 selective inhibitor is rofecoxib.

9. The method according to claim 8 wherein the combination dosage regimen comprises: administering rofecoxib at a dose of 12.5 or 25 mg on a once daily basis and reducing the amount of methotrexate by 2.5 mg per week relative to the DMARD dosing regimen.

10. The method according to claim 4 wherein the DMARD is etanercept.

11. The method according to claim 10 wherein the DMARD dosing regimen is 25 mg twice weekly.

12. The method according to claim 11 wherein the cyclooxygenase-2 selective inhibitor is rofecoxib.

13. The method according to claim 12 wherein the combination dosage regimen comprises: administering rofecoxib at a dose of 12.5 or 25 mg on a once daily basis and administering etanercept at a dose of 25 mg on a once weekly basis.

14. The method according to claim 4 further comprising: eliminating administering the DMARD to the patient and continuing therapy with the cyclooxygenase-2 selective inhibitor alone.

15. A DMARD sparing method in accordance with claim 1 for treating a chronic inflammatory disease or condition in a human patient in need thereof, comprising administering to the patient a therapeutically effective amount of a DMARD in accordance with a DMARD dosage regimen for a period of time, and thereafter administering to the patient a therapeutically effective amount of a cyclooxygenase-2 selective inhibitor in accordance with a COX-2 dosage regimen, whereby the total exposure to the DMARD is reduced.

16. The method according to claim 15 wherein the DMARD is etanercept.

17. The method according to claim 16 wherein the DMARD dosing regimen is 25 mg twice weekly.

18. The method according to claim 17 wherein the cyclooxygenase-2 selective inhibitor is rofecoxib.

19. The method according to claim 18 wherein rofecoxib is administered at a dose of 12.5 or 25 mg on a once daily basis.

20. The method according to claim 1, further comprising co-administering a cyclooxygenase-2 selective inhibitor to the patient being administered the DMARD in accordance with the DMARD dosage regimen, wherein the cyclooxygenase-2 selective inhibitor is administered at a dose which, in combination with the DMARD in accordance with a DMARD dosage regimen, is effective to treat the chronic inflammatory disease or condition.
